(19) United States
(12) Patent Application Publication
(10) Pub. No.: US 2005/0209705 A1
(43) Pub. Date: Sep. 22, 2005

(54) Implant scaffold combined with autologous or allogenic tissue

(76) Inventors: Gabriele G. Niederauer, San Antonio, TX (US); Fred B. Dinger III, San Antonio, TX (US)

Correspondence Address: GREENLEE WINNER AND SULLIVAN P C, 4875 PEARL EAST CIRCLE, SUITE 200, BOULDER, CO, 80301, US

(21) Appl. No.: 11/076,419

(22) Filed: Mar. 9, 2005

Related U.S. Application Data
(60) Provisional application No. 60/551,839, filed on Mar. 9, 2004.

(51) Int. Cl. A61F 2/02

(52) U.S. Cl. 623/23.63; 623/23.72

(57) ABSTRACT

This invention provides implants comprising tissue having an intercellular matrix anchored to a biocompatible scaffold. The intercellular matrix of the tissue provides a natural medium to facilitate the healing and growth of damaged tissue in a patient. The present invention provides methods of treating damaged tissue in a patient by inserting such implants into the damaged tissue. The implants of the present invention include implants comprising allogenic and/or autologous tissue. The tissue may also be acellular.

### CROSS-REFERENCE TO RELATED APPLICATIONS

This application claims the benefit of U.S. Provisional Application No. 60/551,839, filed Mar. 9, 2004, which is incorporated herein to the extent that there is no inconsistency with the present disclosure.

### BACKGROUND OF THE INVENTION

It is known in the art that implants can be inserted into tissue layers, such as bone and cartilage layers, to treat injuries to those tissue layers. One type of implant consists of synthetic material, such as porous biocompatible foams or polymers, for example as disclosed in U.S. Pat. Nos. 4,186,448; 5,607,474; and 5,716,413. An alternative procedure involves inserting plugs of healthy bone or cartilage that are harvested from a healthy area of the patient's body and transplanted into the defect, as disclosed in U.S. Pat. Nos. 5,152,763, 5,919,196, and 6,358,253.

Another material, named AlloDerm® from LifeCell Corp. (One Millennium Way, Branchburg, N.J. 08876-3876), has shown to facilitate healing when implanted into injured tissue. AlloDerm® is donated human dermal tissue that has been decellularized to remove the risk of rejection and inflammation. A proprietary method developed by LifeCell Corp. removes cells from the dermal tissue but leaves the intercellular matrix intact (U.S. Pat. Nos. 5,364,756 and 5,336,616 and published patent application no. 20030035843). The resulting material provides a natural medium for soft tissue and hard tissue repair. AlloDerm® can be freeze dried through a patented process (U.S. Pat. No. 5,364,756) that does not damage the crucial elements of the tissue structure, such as collagens, elastin and proteoglycans, and packaged with a shelf life up to two years. Once AlloDerm® is implanted into a patient, it quickly revascularizes and repopulates with cells from the patient, thereby naturally remodeling into the patient's own tissue. For example, studies show that AlloDerm® is repopulated with chondrocytes when implanted into a chondral defect.

Other allogenic tissues, such as cartilage, tendon, ligament and similar materials, are also useful for implants. The intercellular matrixes of these tissues are processed to preserve the biological structure and composition, but the cells which may cause an immune response are removed. Similarly, autologous tissues are utilized instead of allografts, and the intercellular matrixes processed as described for allografts. Autologous and allogenic tissues may also be used in micronized form.

Previous attempts to deliver such allogenic or autologous tissue to a patient have been limited to pieces of tissue sutured to a defect, glued onto a defect with an adhesive, or chopped up and packed into a defect. These materials are hard to stabilize and fixate into a joint and difficult to maintain in position as the patient resumes activity. Because sheets and micronized particles of tissues are hard to implant effectively, what is needed is an improved delivery or fixation system.

### SUMMARY OF THE INVENTION

The present invention provides a method of inserting an implant into a patient comprising tissue combined with a structurally sound scaffold as a delivery mechanism for implantation. The implant comprises the intercellular matrix of the tissue and can be acellular or have the cells remain intact. In one embodiment, sheets of tissue, which may include allogenic and/or autologous tissue, are attached to a single or multi-phase scaffold base. In another embodiment, minced tissue, which may include allogenic and/or autologous tissue, is loaded onto a porous, polymeric scaffold. In another embodiment, particulated tissue, which may include allogenic and/or autologous tissue, is co-processed with a polymer to form a composite implant.

Porous constructs and polymeric materials suitable for grafts and implants, and which can be used as the scaffolds of the present invention, are well known in the art, such as those developed by OsteoBiologics, Inc., 12500 Network Blvd., Suite 112, San Antonio, Tex., 78249 (U.S. Pat. Nos. 6,514,286; 6,511,511; 6,344,496; 6,203,573; 6,156,068; 6,001,352; 5,977,204; 5,904,658; 5,876,452; 5,863,297; 5,741,329; 5,716,413; and 5,607,474). Polymers suitable for scaffolds of the present invention are also composed of a fiber-reinforced matrix as detailed in U.S. Pat. No. 6,511,511; or a ceramic component for buffering, as detailed in U.S. Pat. No. 5,741,329, to achieve bimodal degradation or to increase mechanical properties as detailed in U.S. Pat. No. 6,344,496.

One embodiment of the present invention provides an implant comprising a delivery scaffold having a distal end, a proximal end and a body. In the present context, “proximal” refers to the end of the implant or scaffold initially oriented closest to the patient's body and the end of the implant that is inserted into a defect. “Distal” refers to the end of the implant or scaffold initially oriented away from the patient's body and the end that faces out from the defect once the implant is inserted. The “body” of the scaffold refers to the middle section of the scaffold between the distal end and proximal end. Preferably the distal end of the implant is approximately level with the surface of the tissue surrounding the defect when the implant is inserted into a defect.

As used herein, the delivery scaffold refers to a structure suitable for insertion into a tissue defect and able to support tissue attached to the scaffold. The delivery scaffold maintains the shape and position of the tissue during healing. The scaffolding is optionally manufactured to have mechanical properties matching those of the tissue into which it is to be implanted. Such properties include, but are not limited to, porosity, strength, stiffness, compressibility, density, elasticity and orientation of pores or fibers. Delivery scaffolds useful with the present invention include scaffolds made from synthetic materials and scaffolds that are transplanted tissue. Where the delivery scaffold is made from synthetic material, it is preferable that the synthetic material is biocompatible and biodegradable.

Examples of synthetic polymers suitable for use with the present invention include, but are not limited to, alpha poly hydroxy acids (polyglycolide (PGA), poly(L-lactide), poly(D,L-lactide), poly(ε-caprolactone), poly(trimethylene carbonate), poly(ethylene oxide) (PEO), polyhydroxybutyrate (PHA), poly(β-hydroxybutyrate) (PHB), poly(β-hydroxyvalerate) (PHVA), poly(p-dioxanone) (PDS), poly(ortho esters), polyhydroxyalkanates, tyrosine-derived polycarbonates, polypeptides and copolymers of the above. Scaffolds of the present invention optionally include porous polymers having fiber reinforcement, a ceramic component, bioactive molecules, such as osteoinductive or chondroinductive growth factors, or combinations thereof.

Delivery scaffolds are also constructed from plastic, metal, ceramic or any sterile material that does not elicit a reaction from the tissue into which the implant is inserted. If the scaffold is made from a material that does not get absorbed by the surrounding tissue, the scaffold may have to be surgically removed after the desired tissue layers have been healed. Implants of the present invention are also constructed from bone plugs, cartilage plugs, or grafts from other types of tissue. These tissue plugs and grafts may be harvested from subjects other than the patient, from tissue banks, or from different parts of the patient's body. One implant of the present invention comprises a bone plug with a sheet of AlloDerm® or other acellular human tissue attached to the distal end of the plug.

Since a majority of biodegradable polymers suitable for implants are inherently hydrophobic, fluids do not easily absorb and penetrate into the implant. The implant of the present invention may also include a surfactant (less than 1% by weight) to further enhance the absorption of fluids, tissue ingrowth and biocompatibility of the material. A surfactant incorporated into the scaffold polymer at the time of manufacture, so that no post-processing is required, has no appreciable detrimental effect on the manufacturing operation or the creation of the scaffold structure. The implant may further include calcium sulfate, tricalcium phosphate or ceramics to modify the mechanical properties of the implant.

In one embodiment, the delivery scaffold comprises a single material layer. In another embodiment, the delivery scaffold comprises a first material layer and an adjacent second material layer, where the first and second material layers have at least one mechanical property which is different. For example, one material layer may have higher porosity to encourage tissue ingrowth while the other material layer has lower porosity to increase the stiffness. In one embodiment, the scaffold comprises a porous fiber-reinforced polymer, where the orientation of the fibers and pores in the first material layer is perpendicular to the orientation of the fibers and pores in the second material layer. In a further embodiment of the present invention, the fibers and pores in the second material layer are oriented parallel to a line extending from the distal end of the scaffold to the proximal end, and the fibers and pores of the first material layer are oriented perpendicular to the distal-proximal direction.

The tissues suitable for the implants of the present invention are tissues comprising an intercellular matrix, sometimes also referred to as an extracellular matrix, including but not limited to dermal tissue, adipose tissue, bone tissue, cartilage tissue, tendons and ligaments. As used herein, an implant comprising a tissue layer is an implant that contains the tissue's intercellular matrix. The intercellular matrix is a complex structure comprising the tissue's native proteins, molecules, fibers, and vascular channels. Implants of the present invention utilize the intercellular matrix of the tissue to increase the ingrowth of the patient's tissue into the implant during healing and to increase the repair of the damaged tissue. The tissue may be human tissue or animal tissue. Preferably the tissue is allogenic, autologous, or a combination thereof. The tissue is optionally acellular. “Acellular” refers to tissue where the cells have been removed leaving the intercellular matrix. Removing the cells from the tissue will reduce or prevent an immune response by the patient's body, including reducing or preventing inflammation and rejection.

In one embodiment, the implant comprises a tissue layer attached to the scaffold. In a further embodiment, the implant comprises a first tissue layer and a second tissue layer. The tissue that makes up the tissue layer, or layers, of the implant does not have to be the same type as the tissue that is being repaired. For example, an implant comprising human adipose tissue may be used to repair a defect in cartilage tissue. In one embodiment, the tissue that makes up the tissue layer or layers includes, but is not limited to, human dermal tissue, adipose tissue, cartilage tissue, bone tissue, ligament tissue or tendon tissue. Preferably the tissue is allogenic, autologous, or a combination thereof. Optionally, the tissue is acellular. Additionally, the tissue that makes up the first tissue layer may be different from the tissue that makes up the second tissue layer. In a specific embodiment of the present invention, the tissue layer is acellular autologous and/or allogenic human dermal tissue, and the first material layer of the scaffold has a porosity and elasticity similar to bone tissue or cartilage tissue.

One embodiment of the present invention provides an implant comprising:
(a) a biocompatible delivery scaffold comprising a distal end, a proximal end, and a scaffold body made of at least one material layer; and (b) a tissue layer comprising a sheet of tissue, wherein said tissue layer is attached to the distal end of said scaffold. By “attached to the distal end of said scaffold” it is meant that a sheet or a cylindrical piece of the tissue is placed on the distal end a single or multi-phase scaffold and affixed to the scaffold using sutures, rivets, adhesives, or other means known in the art. For example, the tissue sheets can be wrapped around the distal end of a mushroom-shaped scaffold and sutured beneath the distal end of the scaffold to fix the tissue in place. Alternatively, the scaffold can have interlocking parts that fixate the tissue sheet to the scaffold when the parts are put together. Ideally, whatever method used to attach the tissue to the scaffold should not result in a rough, protruding or abrasive surface as this is not ideal for implantation into a patient, particularly for implantation into a joint because it may cause damage to surrounding tissue.

A sheet of tissue is a continuous, broad, flat piece of tissue that can be formed into different shapes, including rectangular or circular. In one embodiment, the sheet of tissue can be cut to match the shape and dimension of the distal end of the implant. In another embodiment, the sheet of tissue is larger than the distal end of the implant and covers the distal end and partial sides of the scaffold.

As an alternative to using a sheet of tissue, the tissue is minced, having an average particle size smaller than the mean pore size of the delivery scaffold, and loaded onto a single or multi-phase scaffold. The minced particle size is between about 100 microns and about 400 microns wide, preferably between about 200 microns and 300 microns. The scaffold pores are up to 1 mm wide, more preferably between about 500 microns and about 1000 microns wide. By “loaded onto a scaffold” it is meant the minced tissue is absorbed by, flowed into, or forced into the delivery scaffold and becomes encapsulated within the pores of the scaffold. The loading of the delivery scaffold is preferably done at the time of surgery. The porous scaffold can be fiber reinforced (as described in U.S. Pat. No. 6,511,511) and the primary direction of the fibers, and therefore the pores, can be vertical, horizontal, or in between.

The minced tissue is loaded onto the scaffold using a number of different techniques. Tissue particles can be loaded by immersing the delivery scaffold in a suspension of tissue particles and gently agitating for about two hours. Alternatively, a vacuum-loading method is used, in which the scaffold is immersed in a suspension of tissue particles and a vacuum applied. For clinical ease of use, a double syringe system is set up whereby the scaffold is placed inside one of the syringe barrels and the tissue suspension is forced back and forth between the syringe barrels to infiltrate the scaffold completely. Loading methods done aseptically in an operating room setting are preferable.

Yet another loading technique is to fix the scaffold to the bottom of a centrifuge or microfuge tube and add a suspension of tissue particles. The scaffold and tissue particle mixture is then spun at 200-1000×G for 5 to 15 minutes. Excess solution is decanted and the loaded implant removed for implantation into a patient.

One embodiment of the present invention provides an implant comprising: (a) a biocompatible delivery scaffold comprising a distal end, a proximal end, and a scaffold body having a porous first material layer; and (b) minced tissue loaded onto said scaffold body. Preferably the tissue is dermal tissue, cartilage tissue or bone tissue, and the scaffold body is biodegradable and has a porosity and elasticity similar to bone or cartilage tissue.

In one embodiment of the present invention, the tissue is particulated and co-processed with the polymer of the delivery scaffold to form a composite implant. The composite implant comprises a biocompatible delivery scaffold having a distal end, a proximal end, and a scaffold body comprising a biodegradable polymer containing particulated tissue. Co-processing the tissue with an acceptable solvent, such as DMSO, allows the tissue to be blended with the dissolved polymer and molded into the desired shape. Whereas implants containing minced tissue trap the tissue within the pores of the scaffold, the tissue particles of the composite implant are part of the scaffold polymer itself and do not depend on pore size to determine the amount of tissue within the scaffold.

The composite implant can be porous, fully dense, single phase or multi-phase. In scenarios where the scaffold polymer is biodegradable, the tissue will be released as the polymer degrades. The composite implant can be formed into a variety of sizes and shapes, including a shredded form, and can also comprise bioactive agents such as growth factors, bone marrow, platelet-rich plasma, or other compositions to encourage tissue ingrowth.

### BRIEF DESCRIPTION OF THE DRAWINGS

FIG. 1A shows an implant of the present invention having a first and second tissue layer. FIG. 1B shows an implant having a first and second tissue layer, where the width of the tissue layers is greater than the width of the scaffold.

FIG. 2A shows an implant of the present invention having an inward depression near the distal end of the scaffold. FIG. 2B shows a sheet of tissue covering the implant of FIG. 2A.

FIG. 3A shows a side view of an implant of the present invention having a single tissue layer attached to the scaffold by a suture, a part of which travels along the side of the scaffold in a surface depression. FIG. 3B shows a front view of the implant of 3A. Part of the sutures used to attach the tissue layer to the scaffold travel along the outside of the implant in surface depressions, while other parts of the sutures travel through the implant.

FIG. 4A shows a cross sectional view of an implant of the present invention having a single tissue layer attached to the scaffold through the use of two sutures. A pair of holes extending from the distal end of the scaffold to the proximal end is formed in the scaffold. The sutures are threaded through the holes, looped through a portion of the tissue layer, and threaded back through the holes to the proximal end of the scaffold. FIG. 4B is an exploded view of an implant having a single tissue layer and pre-formed holes through the scaffold for sutures.

FIG. 5A shows an implant of the invention having a single tissue layer attached to the scaffold by two pins inserted through the tissue layer into the scaffold. FIG. 5B shows an implant where the tissue layer is attached to the scaffold by a pin having a barb to prevent the pin from dislodging. FIG. 5C shows an implant where the tissue layer is fixed to the scaffold by a pin attached to strips placed along the surface of the tissue layer.

FIG. 6A shows an implant of the present invention where the scaffold comprises a first material layer where the pores and fibers are arranged horizontally, and a second material layer where the pores and fibers are arranged vertically. FIG. 6B shows a porous implant of the present invention where the outer sections of the scaffold are loaded with minced tissue.

FIG. 7A shows an exploded view of a two-stage implant of the present invention. FIG. 7B shows a two-stage implant where the first material layer is covered by a sheet of tissue and snapped into place in the second material layer.

FIG. 8 illustrates an implant of the present invention having a first and second tissue layer inserted into a defect.

### DETAILED DESCRIPTION

Preferably, the implants of the present invention are approximately cylindrical in shape but may also be rectangular, particularly long rectangular strips, circular, elongated, or irregularly shaped according to the shape of the defect. Implants can be hand-shapeable implants which are moldable into a wide variety of shapes, as described in U.S. Pat. No. 5,716,413. The scaffold may also have a contoured surface, such as concave or convex, to match the contours of the defect. When the implant is cylindrical, the implant has a diameter of between about 1 mm and 50 mm, preferably between about 3 mm and 30 mm, and more preferably between about 10 mm and 25 mm. The height of the implant is between about 2 mm and about 20 mm, preferably between about 3 mm and about 15 mm, more preferably between about 6 mm and about 12 mm. The diameter or width of the tissue layer or layers may be greater than, less than, or the same as the diameter or width of the scaffold body depending on the shape and size needed to fit within the damage tissue.

In one embodiment where the delivery scaffold is approximately cylindrical in shape, the tissue layer is in the form of a circular disc having a diameter slightly less than the diameter of the delivery scaffold to accommodate the thickness of the tissue layer so that none of the tissue gets sheared off when inserted into a defect. The thickness of the tissue is between approximately 1 mm and approximately 2 mm.

In one embodiment, the tissue layer is attached to the delivery scaffold using sutures. It is preferable that the distal surface of the tissue layer present a smooth surface, therefore the sutures should not be present on the surface of the tissue layer. In one embodiment, the sutures enter into the side of the tissue layer beneath the surface of the distal end of the tissue layer, travel through the body of the scaffold, and exit at or near the proximal end of the scaffold. One length of each suture will travel from the distal end of the scaffold toward the proximal end through the interior of the scaffold body, while the other length of the suture will travel along the outside of the scaffold body. Since the outer sides of the scaffold body will likely contact the sides of the defect in the patient, it is preferable that the sides of the scaffold also be smooth. Surface depressions along the surface of the scaffold body, extending from the proximal end of the scaffold to the distal end, provide space for the sutures to travel along the outside of the scaffold without protruding beyond the scaffold surface. As an alternative, one or more channels may be formed in the scaffold body to provide a path for both lengths of the sutures through the interior of the scaffold body.

As an alternative to sutures, the first tissue layer is attached to the scaffold through the use of pins. After the first tissue layer is placed over the distal end of the scaffold, one or more pins are pushed through the first tissue layer into the scaffold body. Optionally the pins have barbs, preferably angled barbs, to prevent pullout of the pins. Additionally, the one or more pins may include thin strips that cover the distal surface of the first tissue layer to help keep the first tissue layer in place. The strips may be a biodegradable material, or a plastic or metal piece that can be removed after healing. Additionally, the pins and sutures may also be biodegradable.

In one embodiment, the tissue layer is a sheet that is larger than the distal end of the scaffold body. The tissue sheet is placed over the distal end of the scaffold body so that the distal end is completely covered. The free edges of the tissue layer sheet are folded toward the proximal end of the scaffold body, and a suture is placed around the tissue sheet and scaffold body near the distal end.

In one embodiment, the tissue sheet covers a mushroom-shaped scaffold. By mushroom-shaped, it is meant that the scaffold is formed with a depression around the scaffold body near the distal end of the scaffold. The diameter of the distal end of the scaffold can be the same, greater or less than the diameter of the rest of the scaffold body. The tissue sheet is placed over the distal end of the scaffold body so that the distal end is completely covered, and the free edges of the tissue layer sheet are folded toward the proximal end of the scaffold into the depression. A suture is placed around the tissue sheet in the depression.

Optionally the tissue sheet is folded over to form a two-ply sheet before attaching to the scaffold. Additionally, the implant may contain a second tissue layer between the tissue sheet and the distal end of the scaffold. The second tissue layer can be one or more additional sheets of tissue, a layer of minced tissue, a layer of scaffold material containing minced tissue, or a composite material made from scaffold material and particulated tissue. Preferably the tissue is allogenic, autologous, or a combination thereof. Optionally, the tissue is acellular.

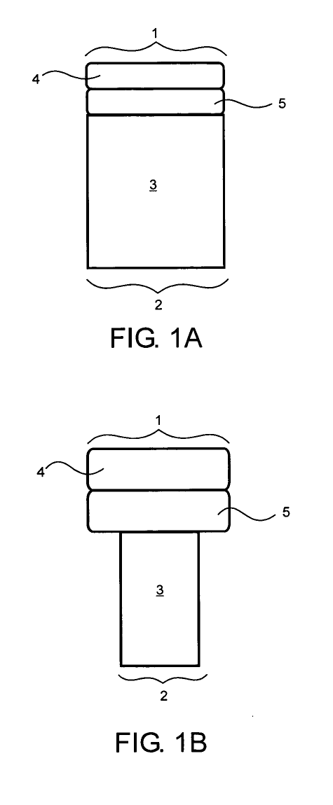

FIG. 1A shows an implant of the present invention comprising a scaffold having a body 3, a distal end 1 and a proximal end 2. In this embodiment, the implant comprises a first tissue layer 4 and a second tissue layer 5 attached to the distal end 1 of the scaffold body 3. The first tissue layer 4 is a cylindrical piece of tissue having the same width or diameter as the scaffold body 3. The second tissue layer 5 is between the first tissue layer 4 and scaffold body 3. The second tissue layer 5 can be a second cylindrical piece of tissue, a layer of scaffold material containing minced tissue, or a composite material made from scaffold material and particulated tissue. In one embodiment, the first tissue layer 4 is cylindrical sheet of acellular human dermal tissue having a thickness between 1 mm and 2mm, and the second tissue layer 5 is a cylindrical heterogeneous layer made from minced acellular human dermal tissue such as Cymetra® (LifeCell Corp., One Millennium Way, Branchburg, N.J. 08876-3876).

FIG. 1B illustrates a similar implant where the first tissue layer 4 and second tissue layer 5 have a width or diameter greater that the width or diameter of the scaffold body 3. Such an implant is useful when the upper area of the defect is larger than lower area of the defect. In one method of the present invention, a hole is drilled into the tissue at the bottom of a defect to provide more room to place the scaffold. The hole drilled into the bottom of the defect is made to have a smaller diameter than the upper portion of the defect in order to minimize the stress on the patient's tissue. The implant illustrated in FIG. 1B would be particularly useful for this method.

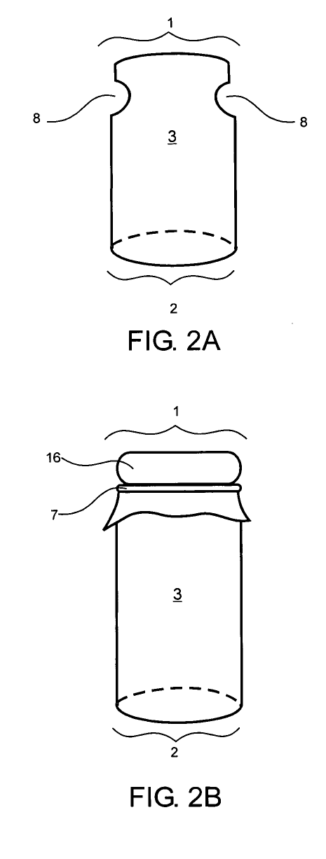

FIG. 2A shows an implant having an annular depression 8 around the scaffold body 3 near the distal end 1. The diameter at the distal end 1 is smaller than the diameter of the rest of the scaffold to accommodate the thickness of the tissue sheet 16. As shown in FIG. 2B, a sheet of tissue 16 is attached to the scaffold by covering the distal end 1 of the scaffold with the sheet of tissue 16 and folding the ends of the sheet of tissue 16 toward the proximal end 2. A suture 7 is used to tie or sew the sheet of tissue 16 to the scaffold body 3 at the annular depression 8 to minimize the portion of the suture 7 which sticks out from the implant.

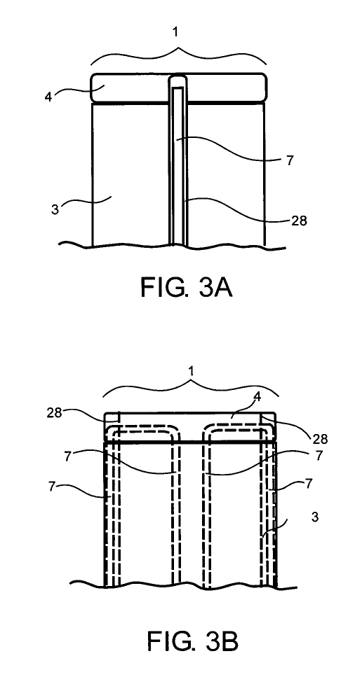

FIGS. 3A and 3B illustrate an alternative method for attaching tissue to a scaffold. A first tissue layer 4 is attached to the scaffold body 3 by a suture 7 which travels along the side of the scaffold body 3 in a surface depression 28. The suture 7 is sewn through the first tissue layer 4 and through the interior of the scaffold body 3.

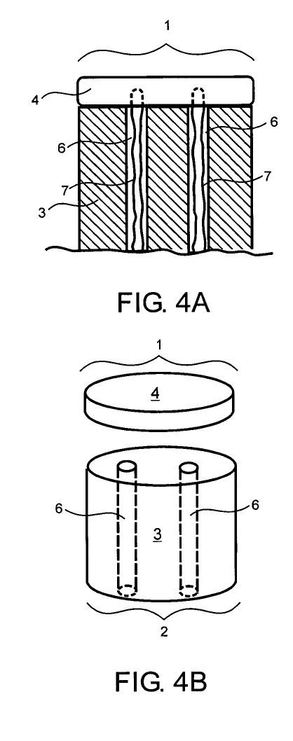

FIGS. 4A and 4B illustrate another method for attaching tissue to a scaffold. Pre-formed channels 6 are formed in the scaffold body 3 which extend from the proximal end (not shown) to the distal end 1. The sutures 7 are threaded through channels 6 in the interior of the scaffold body 3, into the first tissue layer 4, and threaded back through the channels 6. This embodiment is beneficial because it reduces the exposure of the sutures 7 to the surrounding tissue of the patient, thereby reducing irritation and possible inflammation of the surrounding tissue.

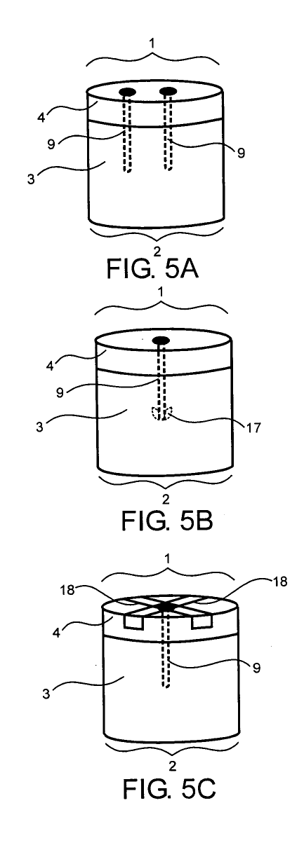

FIGS. 5A, 5B and 5C illustrate another method for attaching tissue to a scaffold. A first tissue layer 4 is attached to a scaffold body 3 by one or more pins 9. The one or more pins 9 are inserted through the first tissue layer 4 and into the scaffold body 3. Optionally, the pins 9 may have barbs 17 (as shown in FIG. 5B) to prevent the pins 9 from being loosened or pulled out of the scaffold body 3. Additionally, multiple pins may be used to provide firm fixation. As shown in FIG. 5C, a pin may optionally have strips 18 on the distal surface of the first tissue layer 4 to further stabilize to position of the first tissue layer 4.

As an alternative to sutures and pins, the tissue layer is attached to the scaffold body using suitable adhesives, as are known in the art. The adhesive is applied to the distal end of said scaffold body and/or the proximal end of the first tissue layer. When the tissue layer is place on the distal end of the scaffold body, the adhesive physically binds the two together. Preferably the adhesive is biocompatible and biodegradable.

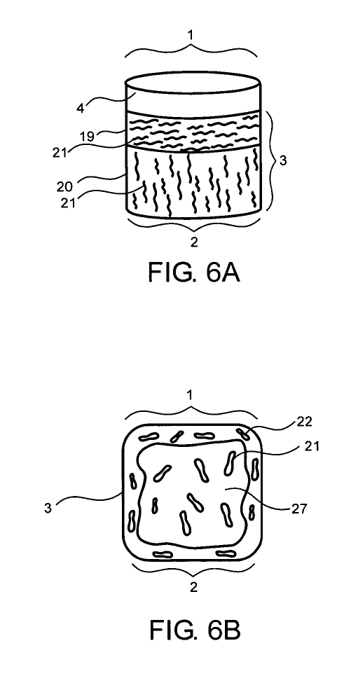

As shown in FIG. 6A, in one embodiment of the invention, the scaffold body 3 comprises a first material layer 19 and a second material layer 20, which differ in at least one mechanical property. Where the scaffold is made from a porous fiber reinforced polymer, the differentiating property may be different orientation and direction of the fibers and pores. FIG. 6A shows an implant having a first material layer 19, where the fiber and pore lattice 21 is oriented perpendicular to the distal-to-proximal direction, and a second material layer 20, where the fiber and pore lattice 21 is orientated parallel the distal-to-proximal direction. The fiber and pore alignment are used to recreate normal hyaline architecture. Normal hyaline cartilage has four layers where the top tissue layers (the layers at or near the joint surface) are parallel to the joint surface to provide better shearing performance and the bottom layers (the layers closest to the bone) are aligned in columnar fashion perpendicular to the surface of the joint.

FIG. 6B illustrates an implant of the present invention comprising a porous fiber reinforced scaffold loaded with minced tissue. The implant comprises a scaffold body 3 having a distal end 1 and a proximal end 2. Placing the scaffold in a suspension of minced tissue and applying a vacuum loads the tissue into the scaffold. The minced tissue will be absorbed into spaces in the fiber and pore lattice 21 of the scaffold and become trapped. FIG. 6B illustrates an implant partially loaded with tissue, where a portion of the scaffold body 3 is loaded scaffold material 22 and a portion is unloaded scaffold material 27. Preferably the entire scaffold is loaded with the tissue. The amount of loaded scaffold material 22 within the scaffold body 3 will depend on the amount of time the scaffold is placed in the vacuum suspension. If the scaffold is placed in the vacuum suspension for longer periods of time, the area of loaded scaffold material 22 will increase.

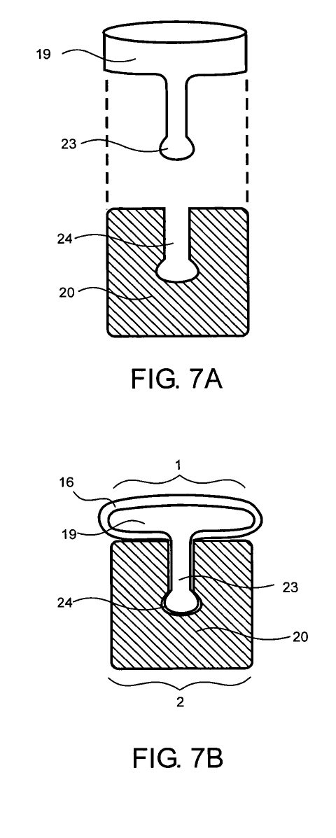

FIGS. 7A and 7B illustrate another implant of the present invention where the scaffold has a snapping mechanism. The scaffold comprises a first material layer 19 and a separate second material layer 20. The first material layer 19 has a snapping attachment 23, and the second material layer 20 has a corresponding receiving cavity 24 suitable for receiving and holding the snapping attachment 23. The length of the snapping attachment 23 corresponds to the depth of the receiving cavity 24 so that when the snapping attachment 23 is inserted in the receiving cavity 24, the proximal surface of the first material layer 19 and the distal surface of the second material layer 20 are in contact. This implant provides another means for attaching a sheet of tissue to a scaffold. As shown in FIG. 7B, a tissue sheet 16 is placed over the distal end 1 of the first material layer 19 with the ends of the tissue sheet 16 folded around the first material layer 19. When the snapping attachment 23 is inserted into receiving cavity 24, the ends of the tissue sheet 16 will be pinned between the first material layer 19 and second material layer 20.

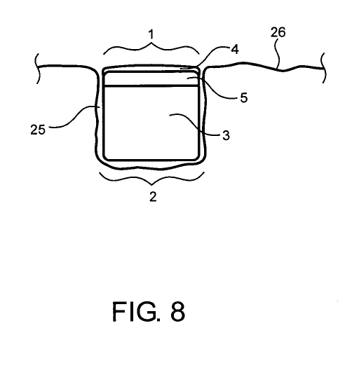

FIG. 8 illustrates an implant of the present invention inserted into a defect 25 in a patient. The implant has a first tissue layer 4 and a second tissue layer 5 attached to a scaffold having a scaffold body 3, a distal end 1 and a proximal end 2. The length of the implant from the distal end to the proximal end should be the same as, or close to, the depth of the defect 25, so that when the implant is inserted into the defect 25, the distal surface of the first tissue layer 4 is approximately level with the surface of the surrounding tissue 26.

A method of promoting regeneration of damaged tissue comprises inserting an implant of the present invention into a defect in damaged tissue. Defects include injuries to a tissue layer of a patient as well as holes intentionally created, such as the hole remaining in bone or cartilage tissue after a plug of healthy bone or cartilage is removed for transplantation. Intentionally created defects also include holes in bone or cartilage tissue created in order to insert autologous, allogenic or synthetic grafts during ligament or tendon repair surgeries. The tissue layer at the distal end of the scaffold provides a smooth articulating surface that enhances integration and healing when in contact with the adjacent tissue. The surface of the tissue layer of the implant should be level with the surface of the surrounding tissue. Preferably the tissue layer, or layers, of the implant is allogenic, autologous, or a combination thereof. Optionally, the tissue is acellular. Tissues that are treatable by implants of the present invention include, but are not limited to, dermal tissue, bone, cartilage, tendons and ligaments. Implants of the present invention can also be used to treat osteochondral defects, particularly those present in joints. The tissue layer of the implant does not have to be the same type of tissue as the defect to be repaired. For example, an implant comprising a tissue layer of acellular dermal tissue is used to repair defects in bone and cartilage tissue.

The defect in the damaged tissue can be intentionally formed or enlarged to accommodate insertion of an implant. For example, a hole can be drilled into the bottom (the portion of the defect furthest away from the surface) of the damaged tissue, so that the depth of the hole is equal to the distance from the proximal end to the distal end of the delivery scaffold. When the implant is inserted into the defect, the scaffold body will fill the drilled hole and the tissue layer of the implant will be approximately level with the surrounding tissue.

While the invention has been described with certain preferred embodiments, it is understood that the preceding description is not intended to limit the scope of the invention. It will be appreciated by one skilled in the art that various equivalents and modifications can be made to the invention shown in the specific embodiments without departing from the spirit and scope of the invention. All publications referred to herein are incorporated herein by reference to the extent not inconsistent herewith.

1. An implant comprising:
(a) a biocompatible delivery scaffold comprising a distal end, a proximal end, and a scaffold body made of at least one material layer; and
(b) a tissue layer comprising a sheet of tissue, wherein said first tissue layer is attached to the distal end of said scaffold.

2. The implant of claim 1 where said tissue is allogenic, autologous, or a combination thereof.

3. The implant of claim 1 where said tissue is acellular.

4. The implant of claim 1 where said tissue is dermal tissue, adipose tissue, cartilage tissue or bone tissue.

5. The implant of claim 1 where said tissue is human tissue.

6. The implant of claim 1 where said material layer has a porosity and elasticity similar to cartilage tissue or bone tissue.

7. The implant of claim 1 where said material layer is a synthetic polymer.

8. The implant of claim 1 where said tissue is acellular human dermal tissue, and said material layer has a porosity and elasticity similar to bone tissue or cartilage tissue.

9. The implant of claim 1 where said material layer is a porous, biocompatible, biodegradable fiber-reinforced polymer.

10. The implant of claim 1 further comprising an adhesive between the distal end of said scaffold body and said tissue layer, wherein said adhesive is in physical contact with the distal end of said scaffold body and said tissue layer.

11. The implant of claim 1 further comprising one or more sutures, wherein each suture is placed through a portion of said tissue layer and through the interior of the scaffold body.

12. The implant of claim 9 further comprising one or more pre-formed channels in said scaffold body extending from the proximal end to the distal end.

13. The implant of claim 1 where said delivery scaffold further comprises an annular depression around the scaffold body near the distal end of said scaffold.

14. The implant of claim 11 further comprising a suture attaching said tissue layer to said annular depression.

15. The implant of claim 1 further comprising one or more pins disposed through said tissue layer and into the scaffold body.

16. The implant of claim 1 further comprising a second tissue layer.

17. The implant of claim 16, wherein said second tissue layer is allogenic, autologous, or a combination thereof.

18. The implant of claim 16 where said second tissue layer is acellular.

19. The implant of claim 1 where said scaffold body comprises a second material layer adjacent to and proximal to said material layer.

20. The implant of claim 19 where said material layers comprise porous, biocompatible, biodegradable fiber-reinforced polymers, where the orientation of the fibers in one material layer is perpendicular to the orientation of the fibers in the other material layer.

21. The implant of claim 19 further comprising a snapping mechanism comprising:
(a) a snapping attachment extending from a surface of one material layer; and
(b) a receiving cavity disposed in the other material layer and extending below the surface thereof, said receiving cavity adapted to receive and hold said snapping attachment;
wherein the length of said snapping attachment is the same as the depth of said receiving cavity, so that when said snapping attachment is fully inserted into said receiving cavity, the said surfaces of both material layers contact each other.

22. An implant comprising:
(a) a biocompatible delivery scaffold comprising a distal end, a proximal end, and a scaffold body having a porous material layer; and
(b) minced tissue loaded onto said scaffold body.

23. The implant of claim 22 where said tissue is dermal tissue, cartilage tissue or bone tissue.

24. The implant of claim 22 where said tissue is allogenic, autologous, or a combination thereof.

25. The implant of claim 22 where said tissue is acellular.

26. The implant of claim 22 where said porous material layer is biodegradable.

27. The implant of claim 22 where said porous material layer has a porosity and elasticity similar to cartilage tissue or bone tissue.

28. An implant comprising a biocompatible delivery scaffold having a distal end, a proximal end, and a scaffold body comprising a composite biodegradable polymer containing particulated tissue.

29. The implant of claim 28 where said particulated tissue is allogenic, autologous, or a combination thereof.

30. The implant of claim 28 where said tissue is dermal tissue, cartilage tissue or bone tissue.

31. The implant of claim 28 where said tissue is acellular.

32. The implant of claim 28 where said scaffold body has a porosity and elasticity similar to cartilage tissue or bone tissue.

33. A method of promoting regeneration of damaged tissue comprising inserting the implant of claim 1 into a defect in said damaged tissue.

34. The method of claim 33 further comprising the step of drilling a hole in the bottom of said defect, where the depth of said hole is equal to the distance from the proximal end to the distal end of said implant.

35. The method if claim 33 wherein the distal surface of said tissue layer is approximately level with the surface of the surrounding native tissue.
